(19) United States
(12) Patent Application Publication
(10) Pub. No.: US 2002/0107535 A1
(43) Pub. Date: Aug. 8, 2002

(54) Union for connection of artificial vessel to human vessel

(76) Inventors: Jeng Wei, Taipei (TW); Tan-Yen Sun, Jang Hua Hsien (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 09/778,726

(22) Filed: Feb. 8, 2001

(51) Int. Cl. A61B 17/11

(52) U.S. Cl. 606/155

(57) ABSTRACT

A union for connection of artificial vessel to human vessel is provided. The union is made of a biocompatible material, such as titanium, for fixedly positioning in an open end of an artificial vessel before a cut human vessel is put around the same open end of the artificial vessel and fixedly connected thereto with a binding string wound around the human vessel against a predetermined location on the union. The union allows the artificial vessel to be quickly connected to the cut human vessel to reduce bleeding and time needed to complete a surgical operation. And, the positioning of the union in the artificial vessel also reduces the difference between an outer diameter of the human vessel and an inner diameter of the artificial vessel at the joint of the two vessels.

### BACKGROUND OF THE INVENTION

In surgical operations for treating an aortic aneurysm and other aortic disease, it is necessary to connect an artificial vessel A to two ends of a cut aorta B to rebuild the blood vessel as shown in FIG. 1. A conventional artificial vessel A is made of woven Dacron that is an elastically stretchable material. In the aortic surgical operation, a predetermined length of artificial vessel A is prepared for connecting to the aorta B by suturing them with a sutural string. However, since a diseased aorta B becomes fragile and tends to be lacerated and separate from the artificial vessel A at the suture lines, and since it takes considerable time to suture the artificial vessel and the human vessel together and the aorta is apt to bleeding seriously during the prolonged surgical operation to adversely affect the treating effect, there is developed an alternative for connecting the artificial vessel and the human vessel together.

In the developed alternative for connecting the artificial vessel A to a human vessel B, a hard coupler A1 is used with the artificial vessel A. Please refer to FIG. 2, a length of artificial vessel A has a first hard coupler A1 fixedly connected to an end before using. After the artificial vessel A is cut to a desired length depending on actual need in the surgical operation, a second hard coupler A1 is sewed onto the other end of the artificial vessel A. Thereafter, the artificial vessel A is connected to two ends of the cut human vessel B, such as the aorta, by putting the ends of the human vessel B around the hard couplers A1 at two ends of the artifice vessel A and winding two binding strings around two ends of the human vessel B against the two hard couplers A1. This alternative way of connecting the human vessel to the hard couplers A1 on the artificial vessel with minding strings reduces time needed to complete the surgical operation, compared with the conventional way of suturing the artificial and the human vessels together.

The hard coupler A1 is made of a rigid plastic material and designed for mounting around an outer surface of the artificial vessel A. To facilitate fixed connection of the hard coupler A1 to the artificial vessel A through sewing, the hard coupler A1 is coated with a layer of soft and loose artificial fabric. The artificial fabric largely increases an overall thickness of the hard coupler A1, disadvantageously resulting in a large difference between an outer diameter of the human vessel and an inner diameter of the artificial vessel. Moreover, the soft and loose artificial fabric of the hard coupler A1 is not able to stop bleeding at the joint of the human vessel and the artificial vessel. Therefore, drug such as protamine must be administered to the patient to neutralize anti-coagulant, such as heparin, in order to stop bleeding.

### SUMMARY OF THE INVENTION

It is therefore a primary object of the present invention to provide an improved union for connection of artificial vessel to human vessel, so as to eliminate disadvantages existing in the conventional connection of artificial and human vessels.

The union according to the present invention is made of a biocompatible material, such as titanium, for fixedly positioning in an open end of an artificial vessel before a cut human vessel is put around the same open end of the artificial vessel and fixedly connected thereto with a binding string wound around the human vessel against a predetermined location on the union. The union enables quick connection of the artificial vessel to the cut human vessel to reduce bleeding and time needed to complete the surgical operation. And, the positioning of the union in the artificial vessel also reduces the difference between an outer diameter of the human vessel and an inner diameter of the artificial vessel at the joint of the two vessels.

The union of the present invention has at least the following advantages:

1. The difference between the inner diameter of the artificial vessel and the outer diameter of the human vessel is reduced.

2. The union has thin wall and therefore does not occupy too much space in the human vessel.

3. The union enables quick connection of the artificial vessel to the human vessel to reduce surgical operation time and bleeding.

4. The union reduces bleeding at joint of the artificial and the human vessels.

5. The union is made of a biocompatible material, such as titanium.

6. The unions may have different diameters for convenient use in surgical operations.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a perspective showing a conventional way of connecting a conventional artificial vessel to a human vessel by suturing the two vessel together;

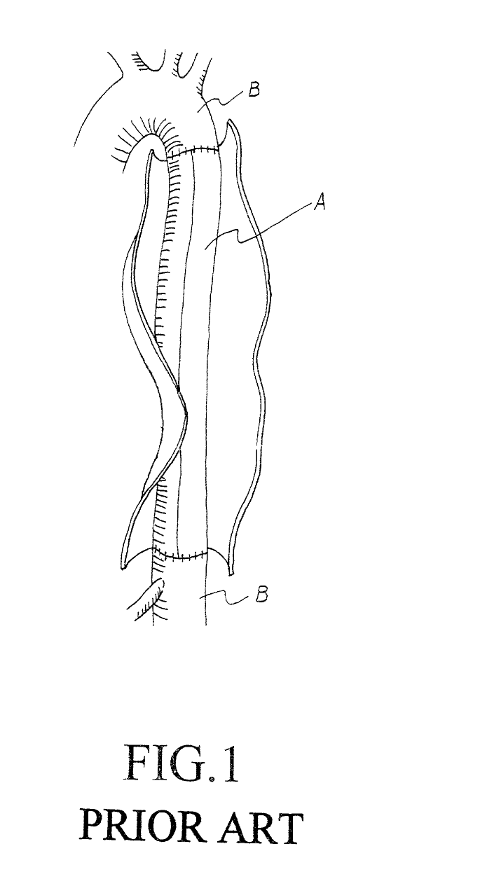

FIG. 2 shows an artificial vessel with conventional hard couplers sewed to two ends thereof;

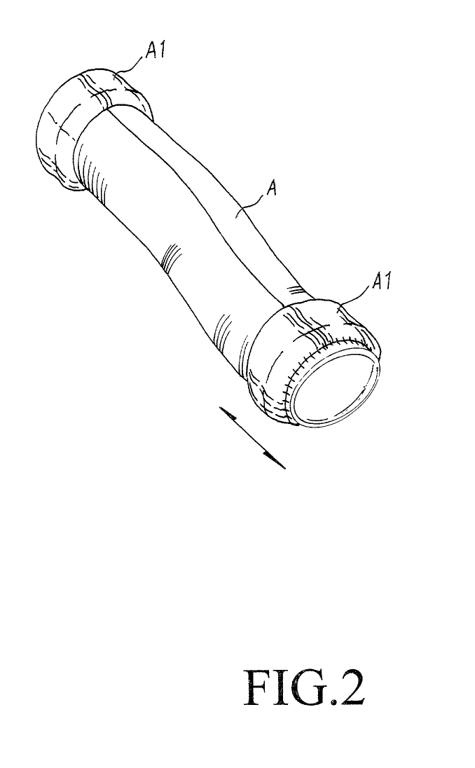

FIG. 3 is a perspective of a union according to a first embodiment of the present invention for connecting an artificial vessel to a human vessel;

FIG. 4 shows a first way of connecting an artificial vessel to a human vessel through the union of the present invention;

FIG. 5 shows another way of connecting an artificial vessel to a human vessel through the union of the present invention;

FIG. 6 is a perspective of a union according to a second embodiment of the present invention for connecting an artificial vessel to a human vessel;

FIG. 7 shows the connection of an artificial vessel to a human vessel through the union of FIG. 6; and

FIG. 8 is a perspective showing the connection of artificial vessels to human vessels through unions of the present invention.

### DETAILED DESCRIPTION OF THE PREFERRED EMBODIMENTS

The present invention relates to a union 1 for connecting artificial and human vessels A and B together. In the following description with reference to the accompanying drawings, similar components shown in different drawings all are denoted by the same reference numeral.

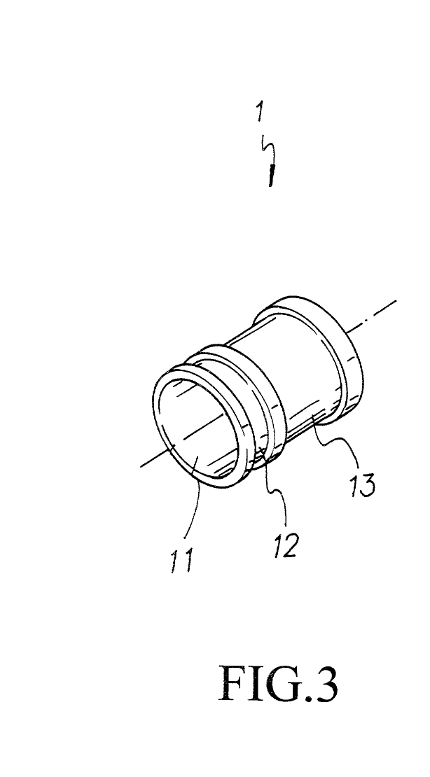

Please refer to FIG. 3 in which a union 1 according to a first embodiment of the present invention is shown. The union 1 is a hollow cylindrical member made of a biocompatible material, such as titanium. The union has a generally smooth inner wall surface 11 and an outer wall surface provided with a small annular groove 12 and a large annular groove 13 that are axially spaced from each other on the union 1.

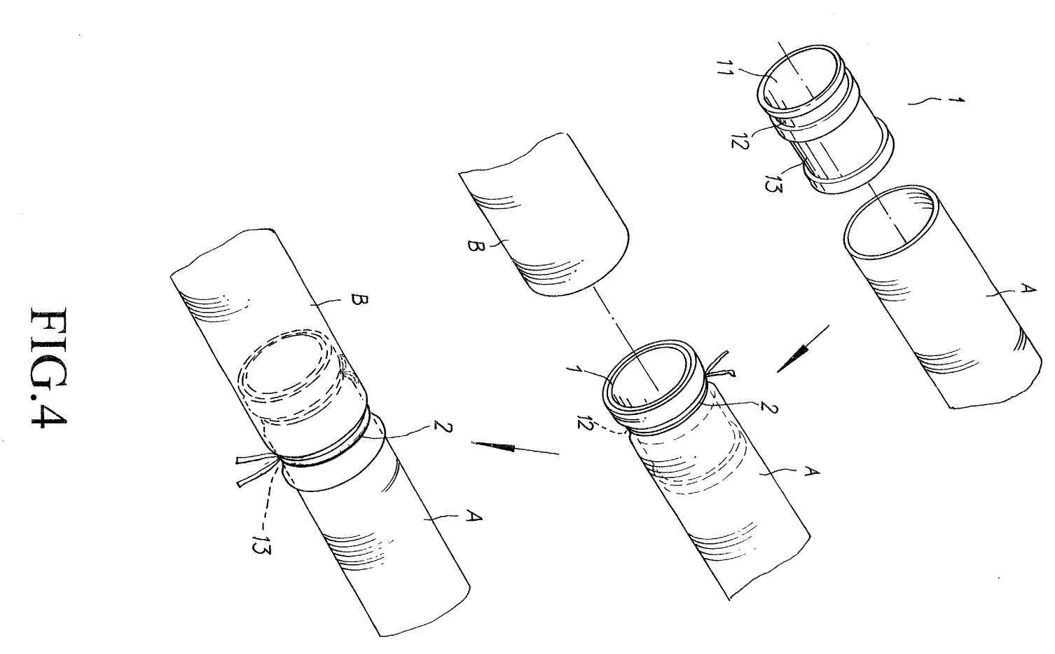

Please refer to FIG. 4 that shows a first way of connecting an artificial vessel A to a human vessel B through the union 1. First, the union 1 is positioned into one open end of the artificial vessel A. A first thinner binding string 2 made of a non-absorbent material compatible with human tissue is used to wind around the open end of the artificial vessel A against the small annular groove 12 of the union 1 so as to bind the artificial vessel A and the union 1 together at the small annular groove 12. Thereafter put an open end of a cut human vessel B around the same end of the artificial vessel A having the union 1 positioned therein and wind a second thicker binding string 2 around the human vessel B against the large annular groove 13 of the union 1 so that the human vessel B is firmly connected to the artificial vessel A within a very short time. Please note the two binding strings 2 are separately wound around the artificial vessel A and the human vessel B at two spaced positions, namely, the small annular groove 12 and the large annular groove 13 of the union 1.

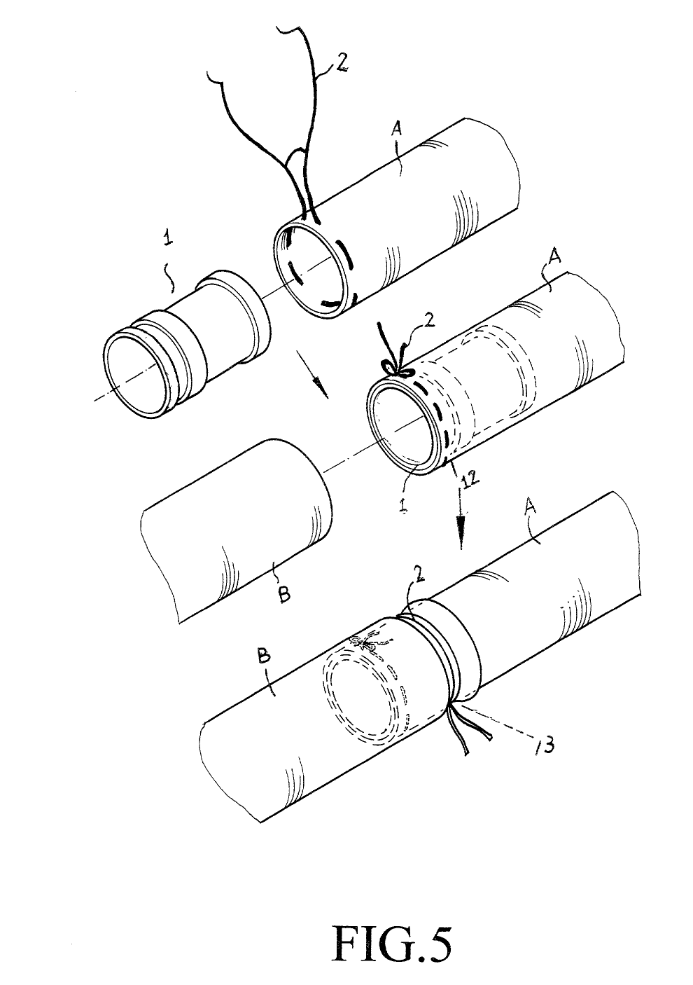

Please refer to FIG. 5 that shows another way of connecting an artificial vessel A to a human vessel B through the union 1. Here, the first thinner binding string 2 may be a non-absorbent sutural string and is circumferentially threaded through a wall of an open end of the artificial vessel A before a surgical operation starts. When the union 1 is put into the open end of the artificial vessel A having the first thinner binding string 2 readily provided thereto, the first thinner binding string 2 could be directly tightened against the small annular groove 12 of the union 1. With the first thinner binding string 2 threaded through the open end of the artificial vessel A in advance, time required to complete the surgical operation can be further shortened.

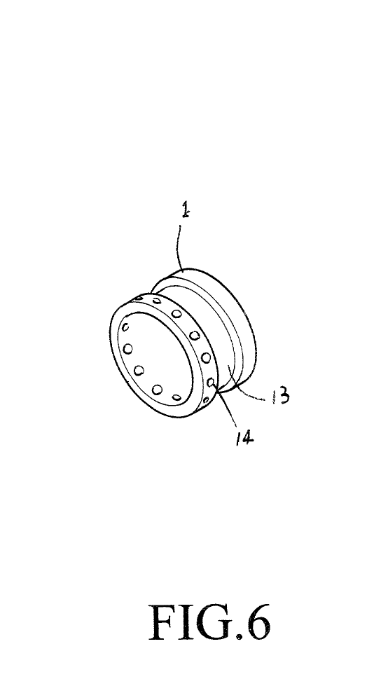

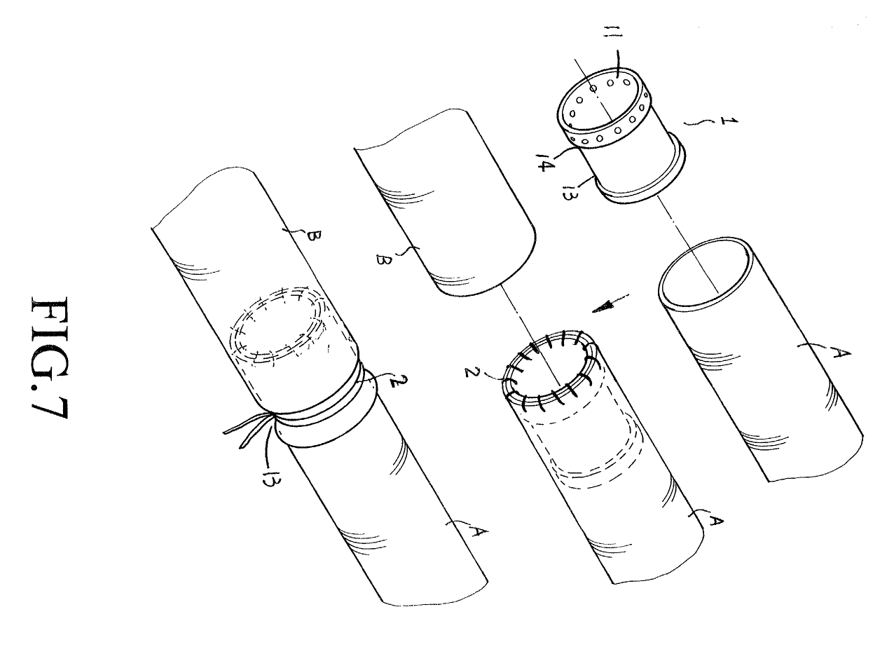

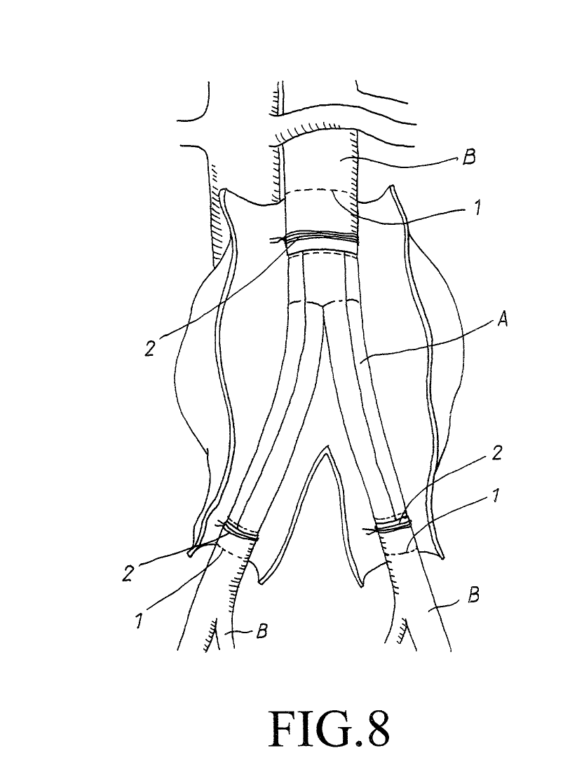

FIG. 6 shows a union 1 according to a second embodiment of the present invention. In this second embodiment, the union 1 is a hollow cylindrical member having a generally smooth inner wall surface 11 and an outer wall surface having two flanges at two ends and a large annular groove 13 between the two flanges. One of the two flanges is provided with a plurality of radially extended and circumferentially arranged through holes 14. FIG. 7 shows the connection of an artificial vessel A to a human vessel B through the union 1 of FIG. 6. In doing so, first position the union 1 in an open end of the artificial vessel A and firmly sew the artificial vessel A and the union 1 together by threading a non-absorbent sutural string 2 through the holes 14 one by one. Thereafter, put an open end of a cut human vessel B around the same open end of the artificial vessel A having the union 1 positioned therein and wind a second binding string 2 around the human vessel B against the large annular groove 13 of the union 1, so that the human vessel B is firmly connected to the artificial vessel. FIG. 8 shows an example of connecting a V-shaped artificial vessel A among three cut human vessels B through the unions 1 of the present invention.

Time required to bind the artificial vessel A and the human vessel B together with a binding string 2 is shorter than that needed to suture the vessels A and B. Moreover, the use of the union 1 at two ends of a cut human vessel B saves the time needed to align two vessels with one another and reduces a distance between the inner wall surface 11 of the artificial vessel A and an outer wall surface of the human vessel B at the connecting point.

The advantages of using the union 1 to connect the artificial vessel A and the human vessel B together include:

1. The union 1 is fixedly positioned in the artificial vessel, allowing the artificial vessel to be more easily fitted into the human vessel.

2. The union 1 may be made of titanium that is excellently biocompatible and has extraordinary hardness that enables the union 1 to have a thin wall and not to occupy too much space in the human vessel.

3. The union 1 reduces the time needed to complete a surgical operation.

4. The artificial vessel between the titanium union and the human vessel is made of Dacron that is blood-impermeable. And, the connection of the artificial vessel to the human vessel through binding does not form tiny sutural holes on the vessels and thereby reduces bleeding at the joint of the vessels.

5. The unions 1 may have various specifications to facilitate application of the unions 1 in surgical operations.

What is claimed is:

1. A union for connection of artificial vessel to human vessel, comprising a hollow cylindrical member in a predetermined length and made of a biocompatible material, said union having a generally smooth inner wall surface and an outer wall surface provided with a small annular groove and a large annular groove, and said small and said large annular grooves being axially spaced on said outer wall surface of said union.

2. A union for connection of artificial vessel to human vessel, comprising a hollow cylindrical member in a predetermined length and made of a biocompatible material, said union having a generally smooth inner wall surface and an outer wall surface having two flanges provided at two ends and an annular groove located between said two flanges, one of said two flanges being provided with a plurality of circumferentially arranged and radially extended through holes, and said union being adapted to fixedly position in one open end of an artificial vessel in advance by threading a binding string through said holes on said flange and sewing said union and said artificial vessel together.
